(19) United States
(12) Patent Application Publication
(10) Pub. No.: US 2003/0060511 A1
(43) Pub. Date: Mar. 27, 2003

(54) Method for treatment of ocular hypertension and glaucoma

(75) Inventor: Ryuji Ueno, Montgomery, MD (US)

Correspondence Address: SUGHRUE MION, PLLC, 2100 Pennsylvania Avenue, NW, Washington, DC, 20037-3213, US

(73) Assignee: SUCAMPO AG

(21) Appl. No.: 10/224,923

(22) Filed: Aug. 22, 2002

Related U.S. Application Data
(60) Provisional application No. 60/314,110, filed on Aug. 23, 2001.

(51) Int. Cl. A61K 31/557

(52) U.S. Cl. 514/573

(57) ABSTRACT

Provided is a method for treating ocular hypertension and glaucoma, which comprises administrating an effective amount of 15-keto-prostaglandin compound having a ring structure at the end of the ω chain to the eyes of a mammalian subject in need of such treatment once a day. According to the method, single administration of the compound effectively lower the IOP of the subject throughout the day.

### TECHNICAL FIELD

The present invention relates to a method for treating ocular hypertension and glaucoma of a mammalian subject.

### BACKGROUND ART

Prostaglandins (hereinafter, referred to as PG(s)) are members of class of organic carboxylic acids, which are contained in tissues or organs of human or other mammals, and exhibit a wide range of physiological activity. PGs found in nature (primary PGs) generally have a prostanoic acid skeleton as shown in the formula (A):

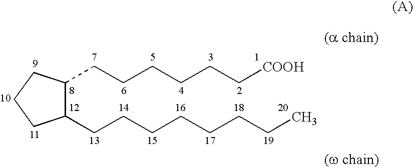

On the other hand, some of synthetic analogues of primary PGs have modified skeletons. The primary PGs are classified to PGAs, PGBs, PGCs, PGDs, PGEs, PGFs, PGGs, PGHs, PGIs and PGJs according to the structure of the five-membered ring moiety, and further classified into the following three types by the number and position of the unsaturated bond at the carbon chain moiety:

Subscript 1: 13,14-unsaturated-15-OH

Subscript 2: 5,6- and 13,14-diunsaturated-15-OH

Subscript 3: 5,6-, 13,14-, and 17,18-triunsaturated-15-OH.

Further, the PGFs are classified, according to the configuration of the hydroxyl group at the 9-position, into α type (the hydroxyl group is of an α-configuration) and β type (the hydroxyl group is of a β-configuration).

PGE₁, PGE₂ and PGE₃ are known to have vasodilation, hypotension, gastric secretion decreasing, intestinal tract movement enhancement, uterine contraction, diuretic, bronchodilation and anti ulcer activities. PGF_{1α}, PGF_{2α} and PGF_{3α} have been known to have hypertension, vasoconstriction, intestinal tract movement enhancement, uterine contraction, lutein body atrophy and bronchoconstriction activities.

PGF_{2α}has a strong affinity with FP receptor, which is one of PG receptors, and has intraocular pressure reducing effects. However, ocular administration of PGF_{2α} or an ester thereof will cause transient IOP increase, and because of such side effects as strong hyperemia in conjunctiva and iris, lacrimation, eye mucus, lid closure, etc., PGF_{2α} cannot be clinically employed.

“Xalatan®” eye drops that has been launched as a pharmaceutical composition for treatment of ocular hypertension and glaucoma contains, as its active ingredient, latanoprost, which is a PG derivative having hydroxy group at the 15-position, i.e., 13,14-dihydro-17-phenyl-18,19,20-trinor-PGF_{2α}-isopropyl ester. Like PGF_{2α}, latanoprost has a strong affinity with the FP receptor and can reduce the IOP throughout the day by ocular administration once a day.

Some 15-keto (i.e., having oxo group at the 15-position instead of the hydroxyl group)-PGs and 13,14-dihydro (i.e., single bond between the 13-position and the 14-position)-15-keto-PGs are the substances naturally produced by the action of enzymes during the metabolism of the primary PGs. It is also known that some 15-keto-PG compounds have IOP reducing effects and are effective for treatment of ocular hypertension and glaucoma (U.S. Pat. Nos. 5,001,153; 5,151,444; 5,166,178 and 5,212,200, all of which are incorporated herein by reference).

It has been known that the 15-keto-PG compound has substantially no affinity with the FP receptor. For example, “Rescula®” eye drops that has been launched as a pharmaceutical composition for treatment of ocular hypertension and glaucoma contains, as its active ingredient, isopropyl unoprostone, which is a metabolic prostaglandin analogue having keto at the 15-position, i.e., 13,14-dihydro-15-keto-20-ethyl- PGF_{2α}-isopropyl ester and has substantially no effect on the FP receptor and other PG receptors. In order to lower the IOP throughout a day, it is necessary to administer isopropyl unoprostone at least twice a day.

In this way, it has been considered to be difficult to provide daylong IOP lowering effect by single administration of the metabolic PG analogue having keto at the 15-position.

Meanwhile, from the viewpoint of the side effects, the present inventor has already found that compounds having substantially or practically no affinity with the FP receptor and other PG receptors, containing PG compounds having keto at the 15-position, cause substantially no iris pigmentation (U.S. Patent Application Publication No. 20020022644) and substantially no ocular irritation such as conjunctival hyperemia or the like. (U.S. Provisional Patent Application No. 60/308,589). These cited references are herein incorporated by reference.

However, “Xalatan®” eye drops containing latanoprost having hydroxy group at the 15-position has a strong affinity with the FP receptor and also has an affinity with other PG receptors such as EP receptor. For this reason, problematic side effects of “Xalatan®” eye drops in clinically applied dose have been reported, including iris pigmentation, ocular irritation such as conjunctival hyperemia and chemosis of conjunctiva (American Journal of Ophthalmology 2001;131:631-635, Survey of Ophthalmology 1997; 41: S105-S110, the cited reference is herein incorporated by reference).

Therefore, in treating ocular hypertension and glaucoma, it has been desired to develop a pharmaceutical composition that can effectively lower the IOP of a subject and keep the low IOP throughout a day by once-a-day administration with substantially no or reduced side effects.

### DISCLOSURE OF THE INVENTION

The present inventor has conducted intensive studies on the biological activity of 15-keto-prostaglandin compounds and has found that a 15-keto-prostaglandin compound having a ring structure at the end of the ω chain can effectively lower the IOP throughout a day in a mammalian subject by administering the same topically to the eyes once a day, and completed the present invention.

Accordingly, the present invention relates to a method for treating ocular hypertension and glaucoma, which comprises administrating an effective amount of a 15-keto-prostaglandin compound having a ring structure at the end of the ω chain to the eyes of a mammalian subject in need of such treatment once a day.

The present invention also relates to an ophthalmic composition for treating ocular hypertension and glaucoma of a mammalian subject, which comprises an effective amount of a 15-keto-prostaglandin compound having a ring structure at the end of the ω chain, wherein said composition is to be administered to the eyes of the subject once a day.

The present invention further relates to use of a 15-keto-prostaglandin compound having a ring structure at the end of the ω chain for manufacturing an ophthalmic composition for treating ocular hypertension and glaucoma of a mammalian subject, wherein said composition is to be administered to the eyes of the subject once a day.

In the present invention, the “15-keto-prostaglandin compound” (hereinafter, referred to as “15-keto-PG compound”) may include any of derivatives or analogs (including substituted derivatives) of a compound having an oxo group at 15-position of the prostanoic acid skeleton instead of the hydroxy group, irrespective of the configuration of the five-membered ring, the number of double bonds, presence or absence of a substituent, or any other modification in the a or ω chain.

The nomenclature of the 15-keto-PG compounds used herein is based on the numbering system of the prostanoic acid represented in the above formula (A).

A preferred compound used in the present invention is represented by the formula (I):

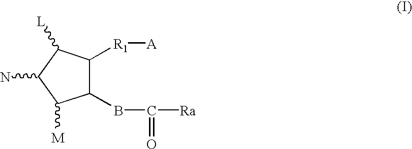

wherein,

L, M and N are hydrogen atom, hydroxy, halogen atom, lower alkyl, hydroxy(lower)alkyl or oxo, wherein at least one of L and M is a group other than hydrogen, and the five-membered ring may have one or more double bonds;

A is —CH₂OH, —COCH₂OH, —COOH or a functional derivative thereof;

B is —CH₂—CH₂—, —CH═CH— or —C≡C—;

R₁ is a saturated or unsaturated bivalent lower or medium aliphatic hydrocarbon residue, which is unsubstituted or substituted with halogen, alkyl, hydroxy, oxo, aryl or heterocyclic group, and one or more carbon atoms in the aliphatic hydrocarbon residue may optionally be replaced by oxygen, nitrogen or sulfur atom; and

Ra is a saturated or unsaturated lower or medium aliphatic hydrocarbon residue, at the end of which is substituted with cyclo(lower)alkyl, cyclo(lower)alkyloxy, aryl, aryloxy, heterocyclic group or hetrocyclic-oxy group.

### BRIEF DESCRIPTION OF THE DRAWINGS

FIG. 1 shows effects of topical application of 0.005% 13,14-dihydro-15-keto-17-phenyl-18,19,20-trinor-PGF_{2α}-isopropyl ester eye drops and 0.005% 13,14-dihydro-17-phenyl-18,19,20-trinor-PGF_{2α}-isopropyl ester eye drops on the IOP in Normal Monkeys: Changes in the IOP from time 0 (ΔIOP) are shown. **ρ<0.01, *ρ<0.05 compared to control (Dunnett's test).

FIG. 2 shows effect of topical application of 0.005% 13,14-dihydro-15-keto-18-phenyl-19,20-dinor-PGF_{2α}-isopropyl ester eye drops on the IOP in Normal Monkeys: Changes in the IOP from time 0 (ΔIOP) are shown.

FIG. 3 shows effect of topical application of 0.005% 13,14-dihydro-15-keto-17-phenoxy-18,19,20-trinor-PGF_{2α}-isopropyl ester eye drops on the IOP in Normal Monkeys: Changes in the IOP from time 0 (ΔIOP) are shown.

### PREFERRED EMBODIMENT OF THE INVENTION

A group of particularly preferable compounds among the above-described compounds is represented by the formula (II):

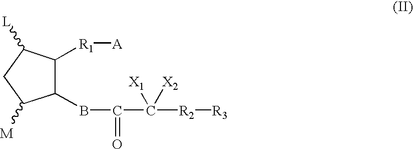

wherein L and M are hydrogen atoms, hydroxy, halogen atoms, lower alkyl, hydroxy(lower)alkyl or oxo, wherein at least one of L and M is a group other than hydrogen, and the five-membered ring may have one or more double bonds;

A is —CH₂OH, —COCH₂OH, —COOH or a functional derivative thereof;

B is —CH₂—CH₂—, —CH═CH—, —C≡C—;

X₁ and X₂ are hydrogen, lower alkyl, or halogen;

R₁ is a saturated or unsaturated bivalent lower or medium aliphatic hydrocarbon residue, which is unsubstituted or substituted with halogen, alkyl, hydroxy, oxo, aryl or heterocyclic group, and one or more carbon atoms in the aliphatic hydrocarbon residue may optionally be replaced by oxygen, nitrogen or sulfur atom;

R₂ is a single bond or lower alkylene; and

R₃ is cyclo(lower)alkyl, cyclo(lower)alkyloxy, aryl, aryloxy, heterocyclic group or heterocyclic-oxy group.

In the above formula, the term “unsaturated” in the definitions for R₁ and Ra is intended to include one or more double bonds and/or triple bonds that are isolatedly, separately or serially present between carbon atoms of the main and/or side chains. According to the usual nomenclature, an unsaturated bond between two serial positions is represented by denoting the lower number of the two positions, and an unsaturated bond between two distal positions is represented by denoting both of the positions.

The term “lower or medium aliphatic hydrocarbon” refers to a straight or branched chain hydrocarbon group having 1 to 14 carbon atoms (for a side chain, 1 to 3 carbon atoms are preferable) and preferably 1 to 10, especially 6 to 10 carbon atoms for R₁ and 1 to 10, especially 1 to 8 carbon atoms for Rₐ.

The term “halogen atom” covers fluorine, chlorine, bromine and iodine.

The term “lower” throughout the specification is intended to include a group having 1 to 6 carbon atoms unless otherwise specified.

The term “lower alkyl” refers to a straight or branched chain saturated hydrocarbon group containing 1 to 6 carbon atoms and includes, for example, methyl, ethyl, propyl, isopropyl, butyl, isobutyl, t-butyl, pentyl and hexyl.

The term “lower alkoxy” refers to a group of lower alkyl-O-, wherein lower alkyl is as defined above.

The term “hydroxy(lower)alkyl” refers to a lower alkyl as defined above which is substituted with at least one hydroxy group such as hydroxymethyl, 1-hydroxyethyl, 2-hydroxyethyl and 1-methyl-1-hydroxyethyl.

The term “lower alkanoyloxy” refers to a group represented by the formula RCO—O—, wherein RCO— is an acyl group formed by oxidation of a lower alkyl group as defined above, such as acetyl.

The term “cyclo(lower)alkyl” refers to a cyclic group formed by cyclization of a lower alkyl group as defined above but contains three or more carbon atoms, and includes, for example, cyclopropyl, cyclobutyl, cyclopentyl and cyclohexyl.

The term “cyclo(lower)alkyloxy” refers to the group of cyclo(lower)alkyl-O-, wherein cyclo(lower)alkyl is as defined above.

The term “aryl” may include unsubstituted or substituted aromatic hydrocarbon rings (preferably monocyclic groups), for example, phenyl, tolyl, and xylyl. Examples of the substituents are halogen atom and halo(lower)alkyl, wherein halogen atom and lower alkyl are as defined above.

The term “aryloxy” refers to a group represented by the formula ArO—, wherein Ar is aryl as defined above.

The term “heterocyclic group” may include mono- to tri-cyclic, preferably monocyclic heterocyclic group which is 5 to 14, preferably 5 to 10 membered ring having optionally substituted carbon atom and 1 to 4, preferably 1 to 3 of 1 or 2 type of hetero atoms selected from nitrogen atom, oxygen atom and sulfur atom. Examples of the heterocyclic group include furyl, thienyl, pyrrolyl, oxazolyl, isoxazolyl, thiazolyl, isothiazolyl, imidazolyl, pyrazolyl, furazanyl, pyranyl, pyridyl, pyridazinyl, pyrimidyl, pyrazinyl, 2-pyrrolinyl, pyrrolidinyl, 2-imidazolinyl, imidazolidinyl, 2-pyrazolinyl, pyrazolidinyl, piperidino, piperazinyl, morpholino, indolyl, benzothienyl, quinolyl, isoquinolyl, purinyl, quinazolinyl, carbazolyl, acridinyl, phenanthridinyl, benzimidazolyl, benzimidazolinyl, benzothiazolyl and phenothiazinyl groups. Examples of the substituent include halogen and halogen substituted lower alkyl group, wherein halogen and lower alkyl group are those as described above.

The term “heterocyclic-oxy group” means a group represented by the formula HcO—, wherein Hc is a heterocyclic group as described above.

The term “functional derivative” of A includes salts (preferably pharmaceutically acceptable salts), ethers, esters and amides.

Suitable “pharmaceutically acceptable salts” include conventionally used non-toxic salts, for example a salt with an inorganic base such as an alkali metal salt (such as sodium salt and potassium salt), an alkaline earth metal salt (such as calcium salt and magnesium salt), an ammonium salt; or a salt with an organic base, for example, an amine salt (such as methylamine salt, dimethylamine salt, cyclohexylamine salt, benzylamine salt, piperidine salt, ethylenediamine salt, ethanolamine salt, diethanolamine salt, triethanolamine salt, tris(hydroxymethylamino)ethane salt, monomethyl-monoethanolamine salt, procaine salt and caffeine salt), a basic amino acid salt (such as arginine salt and lysine salt), tetraalkyl ammonium salt and the like. These salts may be prepared by a conventional process, for example from the corresponding acid and base or by salt interchange.

Examples of the ethers include alkyl ethers, for example, lower alkyl ethers such as methyl ether, ethyl ether, propyl ether, isopropyl ether, butyl ether, isobutyl ether, t-butyl ether, pentyl ether and 1-cyclopropyl ethyl ether; and medium or higher alkyl ethers such as octyl ether, diethylhexyl ether, lauryl ether and cetyl ether; unsaturated ethers such as oleyl ether and linolenyl ether; lower alkenyl ethers such as vinyl ether, allyl ether; lower alkynyl ethers such as ethynyl ether and propynyl ether; hydroxy(lower)alkyl ethers such as hydroxyethyl ether and hydroxyisopropyl ether; lower alkoxy (lower)alkyl ethers such as methoxymethyl ether and 1-methoxyethyl ether; optionally substituted aryl ethers such as phenyl ether, tosyl ether, t-butylphenyl ether, salicyl ether, 3,4-di-methoxyphenyl ether and benzamidophenyl ether; and aryl(lower)alkyl ethers such as benzyl ether, trityl ether and benzhydryl ether.

Examples of the esters include aliphatic esters, for example, lower alkyl esters such as methyl ester, ethyl ester, propyl ester, isopropyl ester, butyl ester, isobutyl ester, t-butyl ester, pentyl ester and 1-cyclopropylethyl ester; lower alkenyl esters such as vinyl ester and allyl ester; lower alkynyl esters such as ethynyl ester and propynyl ester; hydroxy(lower)alkyl ester such as hydroxyethyl ester; lower alkoxy (lower) alkyl esters such as methoxymethyl ester and 1-methoxyethyl ester; and optionally substituted aryl esters such as, for example, phenyl ester, tolyl ester, t-butylphenyl ester, salicyl ester, 3,4-di-methoxyphenyl ester and benzamidophenyl ester; and aryl(lower)alkyl ester such as benzyl ester, trityl ester and benzhydryl ester.

The amide of A means a group represented by the formula —CONR′R″, wherein each of R′ and R″ is hydrogen atom, lower alkyl, aryl, alkyl- or aryl-sulfonyl, lower alkenyl and lower alkynyl, and include for example lower alkyl amides such as methylamide, ethylamide, dimethylamide and diethylamide; arylamides such as anilide and toluidide; and alkyl- or aryl-sulfonylamides such as methylsulfonylamide, ethylsulfonyl-amide and tolylsulfonylamide.

Preferred examples of L and M include hydroxy which has a 5-membered ring structure of, so-called, PGF type.

Preferred A is —COOH, its pharmaceutically acceptable salt, ester or amide thereof.

Preferred B is —CH₂—CH₂—, which provides the structure of so-called, 13,14-dihydro type.

Preferred example of X₁ and X₂ is that at least one of them is halogen, more preferably, both of them are halogen, especially, fluorine that provides a structure of, so called 16,16-difluoro type.

Preferred R₁ is a hydrocarbon residue containing 1-10 carbon atoms, preferably, 6-10 carbon atoms. One or more carbon atoms, preferably one carbon atom on R₁ may optionally be replaced by oxygen, nitrogen or sulfur atom.

Examples of R₁ include, for example, the following groups:

—CH₂—CH₂—CH₂—CH₂—CH₂—CH₂ ,

—CH₂—CH═CH—CH₂—CH₂—CH₂—,

—CH₂—CH₂—CH₂—CH₂—CH═CH—,

—CH₂—C≡C—CH₂—CH₂—CH₂—,

—CH₂—CH₂—CH₂—CH₂—CH(CH₃)—CH₂—,

—CH₂—CH₂—CH₂—CH₂—O—CH₂—,

—CH₂—CH═CH—CH₂—O—CH₂—,

—CH₂—C≡C—CH₂—O—CH₂—,

—CH₂—CH₂—CH₂—CH₂—CH₂—CH₂—CH₂—,

—CH₂—CH═CH—CH₂—CH₂—CH₂—CH₂—,

—CH₂—CH₂—CH₂—CH₂—CH₂—CH═CH—,

—CH₂—C≡C—CH₂—CH₂—CH₂—CH₂—,

—CH₂—CH₂—CH₂—CH₂—CH₂—CH(CH₃)—CH₂—,

—CH₂—CH₂—CH₂—CH₂—CH₂—CH₂—CH₂—CH₂—,

—CH₂—CH═CH—CH₂—CH₂—CH₂—CH₂—CH₂—,

—CH₂—CH₂—CH₂—CH₂—CH₂—CH₂—CH═CH—,

—CH₂—C≡C—CH₂—CH₂—CH₂—CH₂—CH₂—,

—CH₂—CH₂—CH₂—CH₂—CH₂—CH₂—CH(CH₃)—CH₂—

Preferred Ra is a hydrocarbon residue containing 1-10 carbon atoms, more preferably, 1-8 carbon atoms at the end of which is substituted with aryl or aryloxy.

The configuration of the ring and the α- and/or ω chains in the above formula (I) and (II) may be the same as or different from that of the primary PGs. However, the present invention also includes a mixture of a compound having a primary type configuration and a compound of a non-primary type configuration.

Typical example of the compound used in the present invention is a 13,14-dihydro-15-keto-17-phenyl-18,19,20-trinor-prostaglandin F compound, 13,14-dihydro-15-keto-18-phenyl-19,20-dinor-prostaglandin compound, 13,14-dihydro-15-keto-17-phenoxy-18,19,20-trino-prostaglandin compound and their derivatives or analogues.

The 15-keto-PG compound of the present invention may be in the keto-hemiacetal equilibrium by formation of a hemiacetal between hydroxy at position 11 and oxo at position 15.

If such tautomeric isomers as above are present, the proportion of both tautomeric isomers varies with the structure of the rest of the molecule or the kind of the substituent present. Sometimes one isomer may predominantly be present in comparison with the other. However, it is to be appreciated that the 15-keto-PG compounds used in the invention include both isomers. Further, while the compounds used in the invention may be represented by a structure formula or name based on keto-type regardless of the presence or absence of the isomers, it is to be noted that such structure or name does not intend to exclude the hemiacetal type compound.

In the present invention, any of isomers such as the individual tautomeric isomers, the mixture thereof, or optical isomers, the mixture thereof, a racemic mixture, and other steric isomers may be used for the same purpose. Some of the compounds used in the present invention may be prepared by the method disclosed in U.S. Pat. Nos. 5,073,569, 5,166,174, 5,221,763, 5,212,324, 5,739,161 and 6,242,485 (these cited references are herein incorporated by reference).

The term “treatment” used herein includes any means of control such as prevention, care, relief of the condition, attenuation of the condition, arrest of progression of the condition.

The term “a subject in need of such treatment” means a subject who is suffering from a disease in which a reduction in his/her intraocular pressure is desirable, for example, glaucoma and ocular hypertension, or a subject who is susceptible to suffering from such disease as discussed above. The subject may be any mammalian subject including human beings.

According to the present invention, the 15-keto-PG compound defined as above may be formulated as an ophthalmic composition and applied once a day topically to the eyes of a mammalian subject. The ophthalmic composition of the present invention may be any form for topical eye administration used in the ophthalmic field such as eye drops and eye ointment. The ophthalmic composition may be prepared in a conventional manner known to the art.

Eye drops may be prepared by dissolving the active ingredients in a sterile aqueous solution such as saline and buffering solution, or an eye drop composition may be the one provided as a combined powder composition comprising the active ingredient to be dissolved in the aqueous solution before use.

Eye drops such as the ones as described in EP-A-0406791 are preferably used in the present invention (the cited reference is herein incorporated by reference). If desired, additives ordinarily used in conventional eye drops may be added. Such additives may include isotonizing agents (e.g., sodium chloride), buffering agent (e.g., boric acid, sodium monohydrogen phosphate, sodium dihydrogen phosphate), preservatives (e.g., benzalkonium chloride, benzethonium chloride, chlorobutanol), thickeners (e.g., saccharide such as lactose, mannitol, maltose; hyaluronic acid or its salt such as sodium hyaluronate, potassium hyaluronate; mucopolysaccharide such as chondroitin sulfate; sodium polyacrylate, carboxyvinyl polymer, crosslinked polyacrylate.)

The eye drops may be formulated as a sterile unit dose type eye drops containing no preservatives.

Eye ointment may also be prepared in a conventional manner known to the art. For example, it may be prepared by mixing the active ingredient into a base component conventionally used for known eye ointments under a sterile condition. Examples of the base components for the eye ointment include petrolatum, selen 50, Plastibase and macrogol, but not limited thereto. Further, in order to increase the hydrophilicity, a surface-active agent can be added to the composition. The eye ointment may also contain the above-mentioned additives such as the preservatives and the like, if desired.

The amount of administration of the active ingredient used in the present invention may vary according to the sex, age and weight of the subject, symptoms to be treated, effects of treatment to be desired, administration methods period of treatment and the like. Typically, an eye drop composition containing 0.0001%-10% of the active ingredient may be instilled once a day. In the case of using an eye ointment composition, a composition containing 0.0001%-10% of the active ingredient may be applied once a day.

The ophthalmic composition of the invention may contain a single active ingredient or a combination of two or more active ingredients. In a combination of plural active ingredients, their respective contents may be suitably increased or decreased in consideration of their therapeutic effects and safety.

Further, the composition of the present invention may suitably include other pharmacologically active ingredients as far as they do not contradict to the object of the present invention.

According to the present invention, once-a-day administration of the ophthalmic composition of the invention can lower the IOP throughout the day. In addition, as already found out by the present inventors, the above-defined 15-keto prostaglandin compounds cause substantially no iris pigmentation, nor ocular irritation such as conjunctival hyperemia or the like.

The present invention will be described in more detail with reference to the following examples, which, however, are not intended to limit the present invention.

### EXAMPLE 1

1) Test Method

Male cynomolgus monkeys (eight monkeys, body weights 3.0-4.5 kg) were used. To the right eyes of the monkeys, 30 μL/eye of 0.005% 13,14-dihydro-15-keto-17-phenyl-18,19,20-trinor-PGF_{2α}-isopropyl ester eye drops, 0.005% 13,14-dihydro-17-phenyl-18,19,20-trinor-PGF_{2α}-isopropyl ester (latanoprost) eye drops or the vehicle was administered once with an interval of at least seven days, and the IOPs in the respective animals was measured.

2) Measurement of IOP

The animals were retained in the sitting position under systemic anesthesia with intramuscular injection of ketamine hydrochloride (Ketalar® 50, Sankyo Co. Ltd.) 7.5-10 mg/kg and their ocular surfaces were anesthetized with 0.4% oxybuprocaine hydrochloride (Benoxil® 0.4% solution, Santen Pharmaceuticals Co., Ltd.). After that, the IOP was measured with a pneumatonometer (Model 30 Classic, Mentor O & O, Inc.). The IOP was measured before (0 hour) and at 2, 4, 8, 12 and 24 hours after the administration of the test substances.

3) Statistical Analysis

Changes of the IOP from that of time 0 (ΔIOP) obtained in the each test groups were compared to that obtained in the vehicle-administered control group at each measurement times. Statistical analysis was made with the Dunnett's multiple comparison test. Critical rates less than 5% were evaluated to be statistically significant.

4) Result

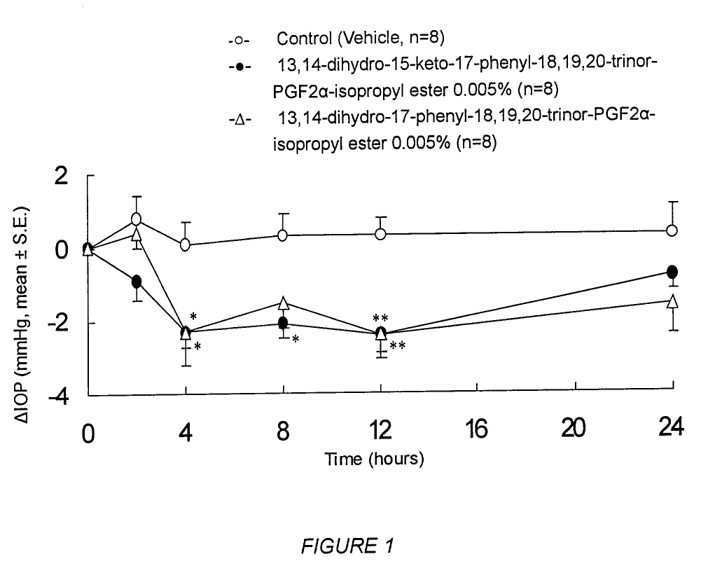

Results were shown in FIG. 1. Single administration of 0.005% 13,14-dihydro-15-keto-17-phenyl-18,19,20-trinor-PGF_{2α}-isopropyl ester eye drops significantly lowered the IOP at 4, 8 and 12 hours after the administration by 2.3±0.4, 2.1±0.4 and 2.4±0.5 mmHg, respectively.

At any time point of the measurement, there were no significant difference between the ΔIOPs of 13,14-dihydro-15-keto-17-phenyl-18,19,20-trinor-PGF_{2α}-isopropyl ester group and those of 13,14-dihydro-17-phenyl-18,19,20-trinor-PGF_{2α}-isopropyl ester (latanoprost) group. Accordingly, the two substances showed similar IOP lowering effect in terms of strength and duration of the effect.

This result indicates that 13,14-dihydro-15-keto-17-phenyl-18,19,20-trinor-PGF_{2α}-isopropyl ester can lower the IOP and keep the low IOP throughout the day by single administration.

### EXAMPLE 2

1) Test Method

Male cynomolgus monkeys (eight monkeys, body weights 3.0-4.5 kg) were used. To the right eyes of the monkeys, 30 μL/eye of 0.005% 13,14-dihydro-15-keto-18-phenyl-19,20-dinor-PGF_{2α}-isopropyl ester eye drops or 0.005% 13,14-dihydro-15-keto-17-phenoxy-18,19,20-trinor-PGF_{2α}-isopropyl ester eye drops were administered, and to the left eyes, 30 μL/eye of saline was administered. IOP of the animals were determined.

2) Measurement of IOP

The animals were retained in the sitting position under systemic anesthesia with intramuscular injection of ketamine hydrochloride (Ketalar® 50, Sankyo Co. Ltd.) 7.5-10 mg/kg and their ocular surfaces were anesthetized with 0.4% oxybuprocaine hydrochloride (Benoxil® 0.4% solution, Santen Pharmaceuticals Co., Ltd.). After that, the IOP was measured with a pneumatonometer (Model 30 Classic, Mentor O & O, Inc.). The IOPs were measured before (0 hour) and at 2, 4, 8, 12 and 24 hours after the administration of test substances.

3) Result

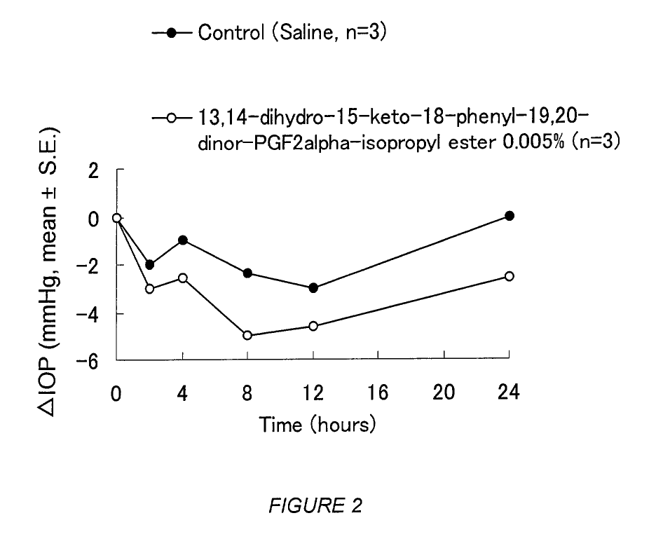

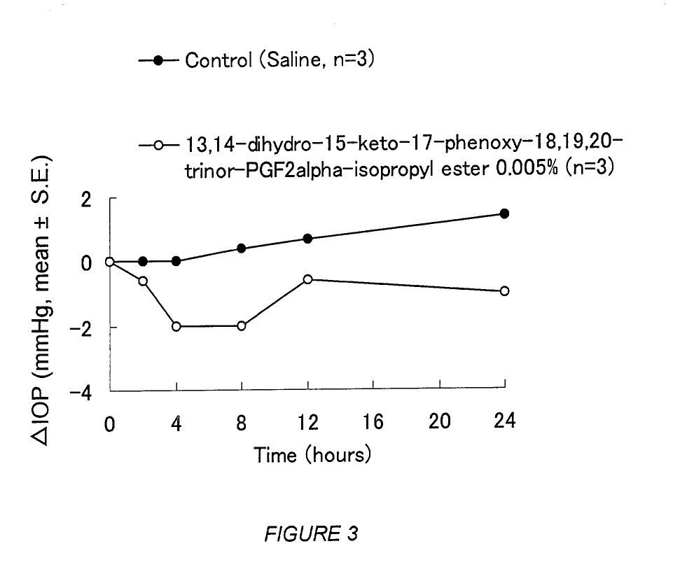

Changes of the IOP from that of time 0 (ΔIOP) obtained in the each test eyes (left eyes) were compared to those obtained in the vehicle administrated eyes (control group) at each measurement times. Results were shown in FIG. 2 and FIG. 3.

These results indicate that both of 13,14-dihydro-15-keto-18-phenyl-19,20-dinor-PGF_{2α}-isopropyl ester and 13,14-dihydro-15-keto-17-phenoxy-18,19,20-trinor-PGF_{2α}-isopropyl ester can lower the IOP and keep the low IOP throughout a day by single administration.

1. A method for treating ocular hypertension and glaucoma, which comprises administrating an effective amount of 15-keto-prostaglandin compound having a ring structure at the end of the ω chain to the eyes of a mammalian subject in need of such treatment once a day.

2. The method as described in claim 1 wherein the 15-keto-prostaglandin compound is a compound represented by the following formula (I): 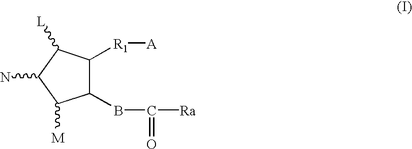
wherein L, M and N are hydrogen atom, hydroxy, halogen atom, lower alkyl, hydroxy(lower)alkyl or oxo, wherein at least one of L and M is a group other than hydrogen, and the five-membered ring may have one or more double bonds;
A is —CH₂OH, —COCH₂OH, —COOH or a functional derivative thereof;
B is —CH₂—CH₂—, —CH═CH— or —C≡C—;
R₁ is a saturated or unsaturated bivalent lower or medium aliphatic hydrocarbon residue, which is unsubstituted or substituted with halogen, alkyl, hydroxy, oxo, aryl or heterocyclic group, and one or more carbon atoms in the aliphatic hydrocarbon residue may optionally be replaced by oxygen, nitrogen or sulfur atom;
Ra is a saturated or unsaturated lower or medium aliphatic hydrocarbon residue, which is substituted at the end with cyclo(lower)alkyl, cyclo(lower)alkyloxy, aryl, aryloxy, heterocyclic group or hetrocyclic-oxy group.

3. The method as described in claim 1 wherein the 15-keto-prostaglandin compound is a 13,14-dihydro-15-keto-prostaglandin compound.

4. The method as described in claim 1 wherein the 15-keto-prostaglandin compound is a 13,14-dihydro-15-keto-17-phenyl-18,19,20-trinor-prostaglandin compound.

5. The method as described in claim 1 wherein the 15-keto-prostaglandin compound is a 13,14-dihydro-15-keto-18-phenyl-19,20-dinor-prostaglandin compound.

6. The method as described in claim 1 wherein the 15-keto-prostaglandin compound is a 13,14-dihydro-15-keto-17-phenoxy-18,19,20-trinor-prostaglandin compound.
